(19) United States
(12) Patent Application Publication
(10) Pub. No.: US 2022/0280177 A1
(43) Pub. Date: Sep. 8, 2022

(54) ELECTRONIC WIRELESS ORAL SUCTION DEVICE

(71) Applicant: Jabril Ahmed Muse, Ilford (GB)

(72) Inventor: Jabril Ahmed Muse, Ilford (GB)

(21) Appl. No.: 17/684,494

(22) Filed: Mar. 2, 2022

Related U.S. Application Data
(60) Provisional application No. 63/155,473, filed on Mar. 2, 2021.

(51) Int. Cl. A61B 17/24; A61B 1/06; A61B 1/24

(52) U.S. Cl. CPC A61B 17/24; A61B 1/06; A61B 1/24; A61B 2017/00734

(57) ABSTRACT

The present invention comprises a body which houses the waterproof vacuum pump, rechargeable battery and the user display interface and a reservoir to hold the aspirated content. Attached to the body of the invention is an extended removable suction head. This portion of the device is used to navigate and aspirate the mouth from unwanted content such as but not limited to tonsil stones, debris and liquids.

### CROSS-REFERENCE TO RELATED APPLICATIONS

This application claims benefit of U.S. Provisional Application No. 63/155,473, filed on Mar. 2, 2021, which is herein incorporated by reference in its entirety.

### FIELD OF THE DISCLOSURE

This invention relates to a novel oral hygiene device.

### BACKGROUND

Oral hygiene devices exist which generate a vacuum usable for extracting solid objects and/or fluids from locations inside a patient's mouth. A conventional wireless or cordless oral vacuum device requires a manual pump, where the suction action is generated by hand operation of the pump. Such devices offer vacuum capability but do not offer an electronic, steady, extended suction action. An oral vacuum device with an electronic pump may offer a steady suction mechanism, but does not offer the benefit of wireless operation. Thus, a need exists for a cordless consumer electronic oral vacuum device, which would address the oral hygiene and medical issues associated with tonsil stones, food debris and fluids. This requirement is currently not satisfied with the devices available.

### SUMMARY

The following is intended to be a brief summary of the embodiments of the invention and is not intended to limit the scope of the invention.

Embodiments of the present invention provide a wireless oral electronic device with an electronic or electrically-powered vacuum action which allows for the aspiration of tonsil stones, fluids and other oral debris. Embodiments of the present invention use a long cylindrical suction head attachment with a narrow diameter to deliver the suction action to the target area in the mouth. On contact, the suction mechanism will engage and draw foreign or native debris and liquids up into the device, where they may travel to a reservoir to be stored. The device may also offer the user a variety of suction strengths. The user will be able to choose their ideal suction strength simply by toggling through the options on a display interface. The device may also include a light to illuminate the target area.

### BRIEF DESCRIPTION OF THE DRAWINGS

A clear understanding of the key features of the embodiments disclosed herein may be had by reference to the appended drawings, which illustrate the method and system of the invention, although it will be understood that such drawings depict particular embodiments of the invention and, therefore, are not to be considered as limiting its scope with regard to other embodiments contemplated by the invention. Accordingly:

FIG. 1 is a profile view of an embodiment of the invention including a suction head, a main body, a light feature underneath the suction head, and a battery charging port situated on the body.

FIG. 2 is a rear perspective view of the embodiment of FIG. 1, showing a user interface of the device.

FIG. 3 is an exploded view of the embodiment of FIG. 1, showing internal components of the invention.

FIGS. 4-6 show additional schematic views of the invention shown in FIGS. 1-3.

### DETAILED DESCRIPTION

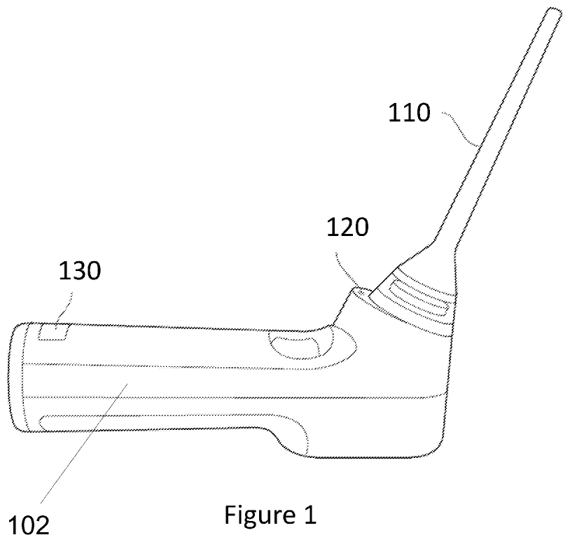

FIG. 1 depicts an example of an electronic wireless oral suction device according to various embodiments described herein. The embodiment shown in the example of FIG. 1 has an elongate tube 110, a light emitter 120 and a charging port 130. The elongate tube 110 may be structured so as to be removable from the remainder of the device. The elongate tube 110 is shaped to be received in the mouth of a user. The tube 110 may also be referred to as a “suction head”. The tube or suction head 110 may be any shape so as to be easily and comfortably received in the mouth of a user. The removable suction head 110 is the only portion of the device that enters the oral orifice. It is used to navigate to the intended target and perform its suction action upon contact with the target. The suction head 110 may be made from food-grade silicone, plastic, rubber, or any other suitable type of material. A handle 102 may be provided to enable a user to grasp the device.

The light emitter 120 of the invention may be situated underneath the suction head 110. In some alternative embodiments, one or more lights 120 can extend around a base of the suction head 110, providing an illuminating ring circling the base of the suction head. The light 120 can provide illumination to facilitate viewing of the interior of a patient's mouth by the user. This improves the user experience of the device.

FIG. 1 also illustrates a charging port 130 for electrical coupling of the device to a power source to recharge the device. In various embodiments, the charging port 130 may be in any of a variety of locations different from the location shown in FIG. 1.

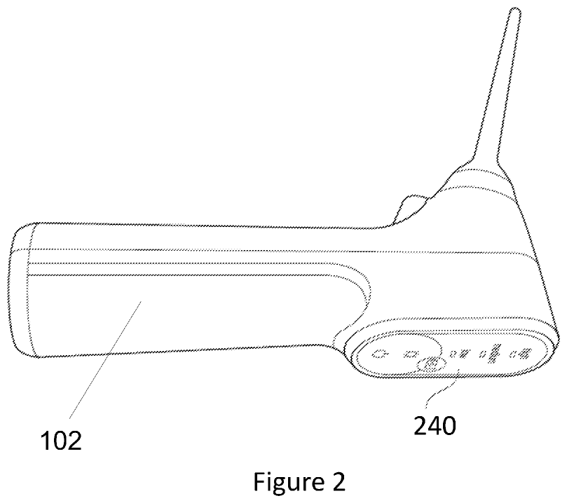

FIG. 2 is a rear perspective view of the embodiment shown in FIG. 1. FIG. 2 depicts a user control interface 240, including a display enabling a user to turn on the light 120 (FIG. 1), monitor battery power and select preferred suction strength. The user may control the power of the vacuum from the interface 240. The interface 240 allows the user to specify the suction strength of the vacuum.

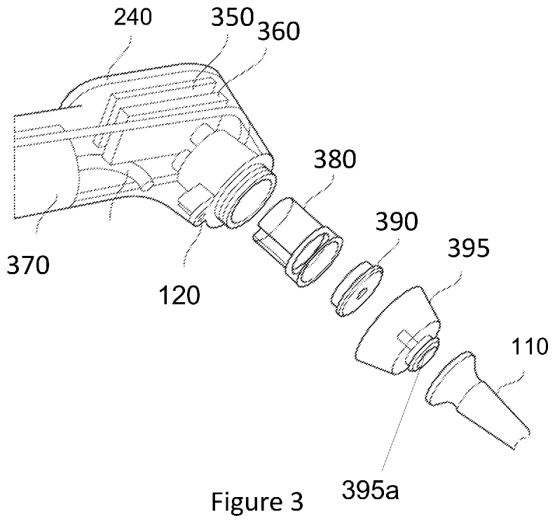

FIG. 3 is an exploded view of the embodiment of FIG. 1. The device may include removable suction head 110, light 120, user interface 240, control circuitry 350 for controlling a waterproof vacuum pump 370, a battery or rechargeable power source 360, a waterproof vacuum pump 370, a reservoir 380, a sealing cap 390 and an aspirator 395. The aspirator 395 may define an inlet 395a structured to receive tonsil stones, fluids and other oral debris drawn into the device by the vacuum. The vacuum pump 370 may be situated in the body of the device. The vacuum pump 370 may be controlled by the control circuitry 350 in conjunction with the interface 240. The pump strength is controlled through the user control interface 240. In one or more arrangements, the maximum pump power may provide suction in the range of around 20 kPa to around 100 kPa. In particular arrangements, the maximum pump power may conform to international suction range 20-60 kPa.

FIG. 3 also illustrates an example of a reservoir 380 which functions as a storage location for the aspirated content as part of various embodiments of the present invention. The reservoir 380 captures material entering the device via the suction head 110. The reservoir 380 can be removable and emptied by a user. The suction head 110 may be of any shape or size to improve ease of use. In this example, the reservoir 380 it is situated at the top of the device as part of the components which connect to the suction head 110. However, the reservoir 380 may be positioned in another location.

FIG. 3 also depicts an internal rechargeable battery 360. In some embodiments, the battery 360 may be a lithium ion battery.

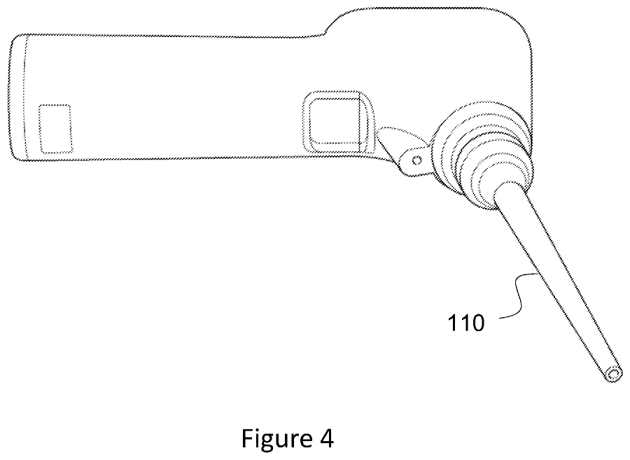

FIG. 4 shows another view of the device with suction head 110.

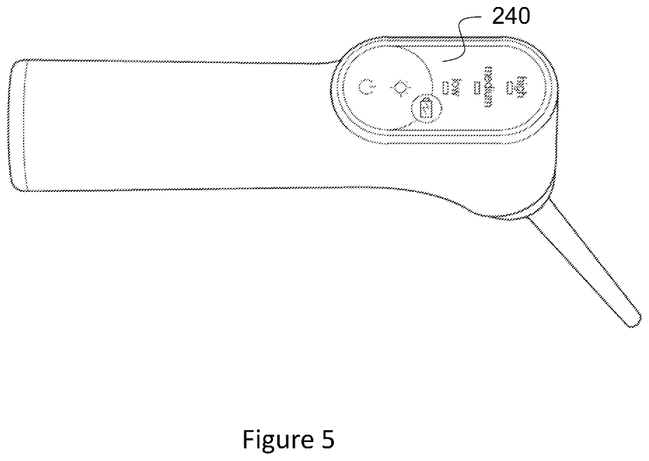

FIG. 5 shows an example of the device with the interface 240. As stated previously, the interface 240 may be used by the user to select different intensities of vacuum. The interface 240 may also be used to turn the device on and off. The interface 240 may be used to activate and deactivate the light.

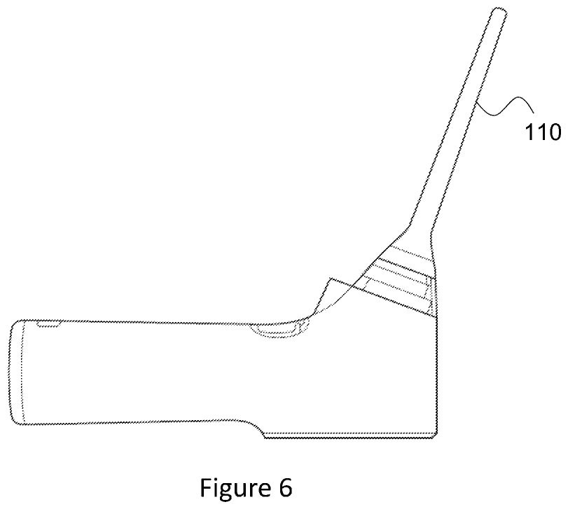

FIG. 6 shows yet another view of the device with suction head 110.

In operation, upon selecting a preferred suction strength using the interface 240 as previously described, the user may manoeuvre the device by grasping the handle 102 and holding the device upright. An angle of the handle 102 with respect to the portion of the device extending from the handle may be different in various embodiments. Once the suction head 110 enters the oral orifice, the suction head 110 may be manipulated to a target area. The path to the target area may be illuminated by the light 120, and on arriving at the location of a target object (for example, a tonsil stone), the user may touch the object with the suction head 110. The object is then immediately sucked up and transported to the reservoir 380.

Cleaning of the components is a simple task. As depicted in FIG. 3, the components can be disassembled and cleaned individually. The embodiments of the present invention are airtight and waterproof and may be run under water without issue.

The terminology used herein is for the purpose of describing particular embodiments only and is not intended to be limiting of the invention. As used herein, the term “and/or” includes any and all combinations of one or more of the associated listed items. As used herein, the singular forms “a,” “an, ” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art pertinent to this invention. It will be further understood that terms, such as those defined in commonly-used dictionaries, should be interpreted as having a meaning that is consistent with their use in the context of the relevant application, and that the present disclosure and is not to be interpreted in an idealized or overly formal sense unless expressly so defined herein.

In describing the embodiments of the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention, and is not intended to limit the invention to the specific embodiments illustrated by the figures or description disclosed herein.

Although the present invention has been illustrated and described herein with reference to preferred embodiments and specific examples thereof, it will be readily apparent to those of ordinary skill in the application that other embodiments and examples may perform similar functions and/or achieve like results. All such equivalent embodiments and examples are within the spirit and scope of the present invention, are contemplated thereby, and are intended to be covered by the following claims.

What is claimed is:

1. An electronic device for use in a mouth of a user, the device comprising:
an electronically controllable vacuum pump arranged to provide a vacuum;
an inlet arranged to provide fluid communication between the electronically controllable vacuum pump and an environment outside of the electronic device; and,
a power source arranged to provide electrical power to the electronically controllable vacuum pump,
wherein the inlet is arranged to receive tonsil stones, fluids and other oral debris.

2. A device according to claim 1, wherein the electronic device is wireless.

3. A device according to claim 1, wherein the device comprises a charging port arranged for use in recharging the power source.

4. A device according to claim 1, wherein the device further comprises an elongate tube removably connectable to the inlet.

5. A device according to claim 4, wherein the elongate tube is a removable suction head.

6. A device according to claim 4, wherein the elongate tube is formed of food-grade silicone, plastic, or rubber.

7. A device according to claim 1, further comprising a user control interface arranged to provide control over the electronically controllable vacuum pump.

8. A device according to claim 7, wherein the electronically controllable vacuum pump is arranged to provide a suction in a range of 20 kPa to 100 kPa.

9. A device according to claim 1, further comprising a reservoir for receiving and storing material passing through the inlet in use.

10. A device according to claim 1, further comprising a handle for use by a user.

11. A device according to claim 1, further comprising a light emitter for emitting light.
